(19) United States
(12) Patent Application Publication
(10) Pub. No.: US 2011/0060392 A1
(43) Pub. Date: Mar. 10, 2011

(54) Implantable Microstimulators

(76) Inventors: Mark Zdeblick, Portola Valley, CA (US); Hooman Hafezi, Redwood City, CA (US)

(21) Appl. No.: 12/601,180

(22) PCT Filed: Nov. 13, 2009
(86) PCT No.: PCT/US09/64474

§ 371 Date: Nov. 20, 2009

Related U.S. Application Data
(62) Division of application No. 61/114,437, filed on Nov. 13, 2008.
Application No. 61/114,437 is a division of application No. 61/115,887, filed on Nov. 18, 2008.

(51) Int. Cl. A61N 1/00

(52) U.S. Cl. 607/115

(57) ABSTRACT

Electrolytic capacitor microstimulators are provided. Microstimulators of the invention are configured to operate, upon implantation into a living body, as an electrolytic capacitor that employs body fluid as its electrolyte. Also provided are methods and systems that include the microstimulators of the invention, as well as methods of using the devices and systems in a variety of different applications.

### RELATED APPLICATION AND CROSS REFERENCE

This application claims the benefit of U.S. Provisional Application Ser. No. 61/114,437 filed on Nov. 13, 2008 and Application Ser. No. 61/115,887 file don Nov. 18, 2008; the entire disclosures of which are incorporated herein by reference.

### FIELD OF THE INVENTION

The present invention relates to compact electronic devices and, more specifically to hermetically sealed devices that provide electrical stimulation to a target site.

### BACKGROUND

Neurostimulators are used to deliver neurostimulation therapy to patients to treat a variety of symptoms or conditions such as chronic pain, tremor, Parkinson's disease, epilepsy, incontinence, or gastroparesis. Implantable neurostimulators may deliver neurostimulation therapy in the form of electrical pulses via implantable leads that include electrodes. To treat the above-identified symptoms or conditions, implantable leads may be implanted along nerves, within the epidural or intrathecal space of the spinal column, and around the brain, or other organs or tissue of a patient, depending on the particular condition that is sought to be treated with the device.

With respect to implantable leads, several elements such as conductors, electrodes and insulators may be combined to produce a lead body. A lead may include one or more conductors extending through the length of the lead body from a distal end to a proximal end of the lead. The conductors electrically connect one or more electrodes at the distal end to one or more connectors at the proximal end of the lead. The electrodes are designed to form an electrical connection or stimulus point with tissue or organs. Lead connectors (sometimes referred to as terminals, contacts, or contact electrodes) are adapted to electrically and mechanically connect leads to implantable pulse generators or RF receivers (stimulation sources), or other medical devices. An insulating material may form the lead body and surround the conductors for electrical isolation between the conductors and for protection from the external contact and for ensuring compatibility with a body.

Such leads may be implanted into the body at an insertion site and extend from the implant site to the stimulation site (area of placement of the electrodes). The implant site may be a subcutaneous pocket that receives and houses the pulse generator or receiver (providing a stimulation source). The implant site may be positioned a distance away from the stimulation site, such as near the buttocks or any other place in the body (e.g., torso area). One common configuration is to have the implant site and insertion site located in the lower back area, with the leads extending through the epidural space in the spine to the stimulation site, such as middle back, upper back, neck or brain areas.

Current lead designs have different shapes, such as those commonly known as percutaneous and paddle-shaped leads. Paddle leads, which are typically larger than percutaneous leads, are directional and often utilized due to desired stimulus effect on the tissues or areas. However, current paddle-shaped leads require insertion using surgical means, and hence, removal, when needed, through surgical means.

Percutaneous leads are designed for easy introduction into the epidural space using a special needle. Therefore, such leads are typically smaller than paddle-shaped leads and more nearly circular in cross-section. This reduced size facilitates their implantation in the body, allows their implantation into more areas of the body, and minimizes the unwanted side effects of their implantation.

### SUMMARY

Electrolytic capacitor microstimulators are provided. Microstimulators of the invention are configured to operate, upon implantation into a living body, as an electrolytic capacitor that employs body fluid as its electrolyte. Also provided are methods and systems that include the microstimulators of the invention, as well as methods of using the devices and systems in a variety of different applications.

### BRIEF DESCRIPTION OF THE FIGURES

FIG. 1 illustrates a microstimulator according to a first embodiment of the invention.

FIG. 2 illustrates a microstimulator according to a second embodiment of the invention.

FIG. 3 illustrates a microstimulator according to a third embodiment of the invention.

### DETAILED DESCRIPTION

Electrolytic capacitor microstimulators are provided. Microstimulators of the invention are configured to operate, upon implantation into a living body, as an electrolytic capacitor that employs body fluid as its electrolyte. Also provided are methods and systems that include the microstimulators of the invention, as well as methods of using the devices and systems in a variety of different applications. The microstimulators in accordance with the present invention may also include an exposed electrode. The exposed electrode may be fabricated from any convenient material, including but not limited to platinum alloys, such as platinum-iridium, which may or may not be coated, such as with a titanium-nickel coating. The electrodes may have any convenient configuration, so long as they are exposed and capable of activation to apply electrical stimulation to a target tissue.

As summarized above, the microstimulators are electrolytic capacitor microstimulators. As the microstimulators of the invention are electrolytic capacitor microstimulators, the microstimulators are configured to operate, upon implantation into a living body, as an electrolytic capacitor that employs tissue and body fluid as its electrolyte. As such, upon implantation, fluid is employed as part of a capacitor unit of the microstimulator, and specifically as the electrolyte of the capacitor unit of the microstimulator.

In some aspects, microstimulators include a hermetically sealed integrated circuit component, a single capacitive plate and a single exposed electrode, where a capacitive plate and an electrode, which is a second capacitive plate, are conductively coupled to the integrated circuit component. Integrated circuit components of the present invention include circuitry components and a solid support (e.g., housing structure). The solid support may be small, for example where it is dimensioned to have a width ranging from 0.01 mm to 100 mm, such as from 0.1 mm to 20 mm, and including from 0.5 mm to 2 mm; a length ranging from 0.01 mm to 100 mm, such as from 0.1 mm to 20 mm, and including from 0.5 mm to 2 mm, and a height ranging from 0.01 mm to 10 mm, including from 0.05 mm to 2 mm, and including from 0.1 mm to 0.5 mm. The solid support element may take a variety of different configurations, such as but not limited to: a chip configuration, a cylinder configuration, a spherical configuration, a disc configuration, etc. A particular configuration may be selected based on intended application, method of manufacture, etc. While the material from which the solid support is fabricated may vary considerably depending on the particular device, in certain aspects the solid support is made up of a semiconductor material, such as silicon.

Microstimulators of the invention may be configured for stimulating a variety of different types of tissue, including but not limited to nervous tissue, muscle tissue, etc. As such, they may be structured specifically for stimulation applications, in terms of device form factor or shape, as well as control unit programming. Devices of the invention may be configured specifically for neural stimulation applications. Such devices may have a variety of shapes that are suitable for use in neural stimulation applications. Programming (a set of instructions that are implemented by a processor to perform a given task) that is specific for a stimulation protocol of interest, such as a neural stimulation protocol or muscle tissue stimulation protocol may also be included in components of devices of the invention, such as integrated circuit elements of the integrated circuit controller and/or individually addressable satellite electrode structures of the devices, as reviewed in greater detail below. Programming that may be part of the devices may include a full set of instructions for a given task or a partial set of instructions that is employed in conjunction with other instructions associated with components distinct from the devices, where such additional instructions may be present in an extracorporeal control unit with which the device communicates at some time associated with operation of the device, e.g., before, during or after the device stimulates target tissue of interest. Microstimulators of the invention are configured to apply an electrical stimulation that is sufficient for an intended purpose, where the stimulation may vary. In some aspects, the microstimulators are configured to stimulate with a voltage ranging from 0 to 10 V, such as 0 to 5 V and including 0 to 1 V.

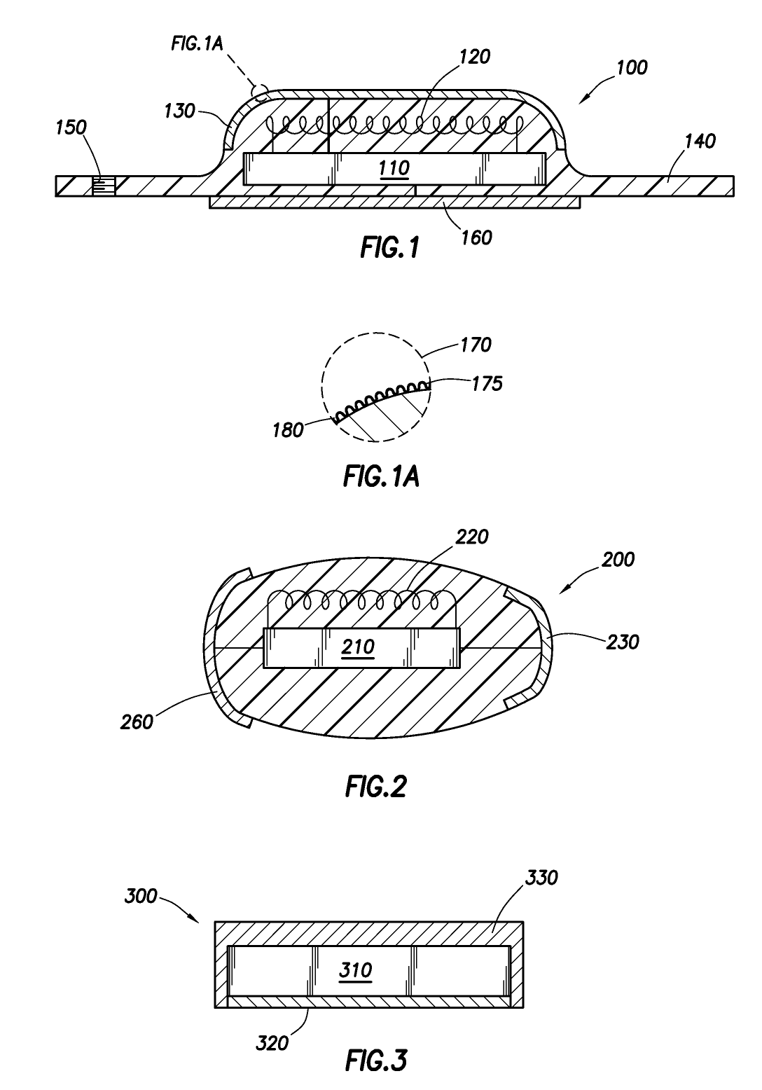

Referring now to FIG. 1, a side view of a planar microstimulator 100 is shown. Microstimulator 100 includes an integrated circuit 110 and a coil 120, which serves as an inductive power source. These components are hermetically sealed. The inductive power source is a component configured to receive power signals, e.g., in the form of radiofrequency energy, from an extracorporeal location, and convert the received signals into energy sufficient to power the integrated circuit 110. The inductive power source may take any convenient shape. In some aspects, the inductive power source is the coil 120. Coils employed in inductive power sources may vary, from loose coils to tight coils, as desired depending on the particular configuration. The inductive power sources may be positioned at any convenient location, including the coil 120 on top of the integrated circuit 110 or in an alternative embodiment, as part of the integrated circuit 110 (not shown).

In one embodiment, a capacitive plate 130 of the microstimulator 100 includes a single capacitive plate, which may also be referred to as an inner capacitive plate. The capacitive plate 130 may have a planar or non-planar configuration, as desired, and may be shaped to conform to other components of the microstimulator 100, such as the integrated circuit 110. Selected portion 170 shows a magnified view of the capacitive plate 130. The magnified view 170 shows the high surface area layer 175 coated with a thin dielectric layer 180.

In some aspects, the capacitive plate 130 includes a surface that has a high surface area. Of interest are plates that have a bottom layer made of a continuous metal and a highly porous top layer, such as layer 180. The capacitor plate 130 may be fabricated from any convenient material, including but not limited to platinum alloys, such as platinum-iridium, which may or may not be coated, such as with a titanium-nickel coating. The capacitor plate 130 may have any convenient configuration. The highly porous top layer may be produced using any convenient protocol, including depositing small metallic spheres onto a metallic surface and then subjecting the spheres to conditions in terms of temperature and/or pressure, sufficient to produce a highly porous coating layer from the deposited spheres. In some aspects, high surface areas as described in PCT application serial no. PCT/US2008/053999 published as WO/2008/101107 (the disclosure of which is herein incorporated by reference) are employed. The coating of the surface of the capacitor also increases the area of the surface by making the top surface shape undulating in an irregular or random fashion. This result can be achieved by Cathodic Arc Deposition of Tantalum and Tantalum Oxide (e.g., Tantalum Pentoxide). Tantalum oxide is Ta2O5, also known as tantalum pentoxide. Both orthorhombic and hexagonal phases are known. Tantalum oxide is a high refractive index, low absorption material useful for coatings in the near-UV to IR spectra regions. Tantalum oxide decomposes only at temperatures >1470° C. Tantalum oxide is used to make capacitors in automotive electronics, cell phones, and pagers, electronic circuitry, thin-film components, and high-speed tools.

The capacitive plate 130 with the layer 180 may be configured such that upon implantation in a body, the capacitive plate 130 is separated from body fluid by the layer 180. Thus, the capacitive plate 130 is configured as a single plate capacitor plate. The thickness of the layer 180 may vary, so long as it is not so thick as to prevent the capacitor plate 130 from functioning for its intended purpose. In some cases, the layer 180 has an average thickness of 5 μm or less, such as 3 μm or less, in some cases 1 μm or less, including 0.1 μm or less, for example 0.05 μm or less. Of interest are dielectric layers having an average thickness ranging from 0.01 to 5 μm to, such as from 0.01 to 3 μm, including from 0.05 to 1 μm, for example 0.1 to 0.5 μm. Any convenient dielectric may be provided, where materials of interest include but are not limited to: nitrides, such as silicon nitride and silicon carbide, oxides, such as titanium oxide and tantalum oxide, and the like. In some aspects, the layer 180 of the capacitive plate 130 is part of a hermetic sealing element, described in greater detail below. In some aspects, the capacitor plate 130 is the first electrode layer of a transition metal coated with a dielectric layer of an oxide of the transition metal, such as titanium dioxide, tantalum oxide, etc. The layer 180 may be configured to provide one or more desired functions. In some aspects, the layer 180 is configured to protect the capacitor plate 130 from corrosion. In some aspects, the layer 180 is configured to prevent DC current from entering the body. In some aspects, the layer 180 allows for the use of higher voltage stimulation by preventing water breakdown.

Continuing with FIG. 1 and according to an aspect of the present invention, the microstimulator 100 is an implantable device that is configured to deliver electrical stimulation to a target tissue. The nature of the electrical stimulation may vary greatly, as further described below. As such, the microstimulator is configured to maintain functionality when present in a physiological environment, including a high salt, high humidity environment found inside of a body. Implantable devices in accordance with the teaching of the present invention are configured to maintain operation and functionality under these conditions over varying periods of time, including, for two or more days, such as one week or longer, four weeks or longer, six months or longer, one year or longer, including five years or longer. The duration of operation does not limit the scope of the present invention as set forth in the claimed invention below. In some aspects, the implantable devices are configured to maintain functionality when implanted at a physiological site for a period ranging from one to eighty years or longer, such as from five to seventy years or longer, and including for a period ranging from ten to fifty years or longer.

One embodiment, as shown in FIG. 1, a non-conductive housing structure 140 is shown that includes a hole 150. The housing 140 effectively increases the distance between the capacitive plate 130 and a capacitive plate 160. The housing 140 is a non-electrically conductive element (such as an electrically insulating layer) that may be configured to provide for a number of different desirable functionalities. In some aspects, the housing 140 is configured to effectively lengthen the distance between the capacitor plate 130 and 160, which results in an increased distance between capacitor and the electrode of the stimulator, respectively. The housing 140 may have a variety of different configurations, so long as it serves to lengthen the distance between the capacitor plates 130 and 160. In some aspects, a configuration is chosen for the housing 140 that focuses energy in a particular manner. Such focusing may also be configured to prevent stimulation of non-target tissue (in other words tissue whose stimulation is not desired). Focusing elements, such as the housing 140, may also be configured to reduce overall power needed for a desired stimulation event. This element may have a two-dimensional or three-dimensional configuration, and have any convenient shape, such as square, disc, triangular, ovoid, irregular, etc., as desired. The distance that the edge of this element may extend beyond the edges of plate 130 or plate 160 may vary, and in certain aspects is 0.05 mm or more, e.g., 0.1 mm or more, including 1.0 mm or more, such as 5.0 mm or more and including 10 mm or more, where the distance may not exceed 100 mm in certain aspects. The housing 140 may be fabricated from a number of different materials, such that it may be made of a single material or be a composite of two or more different types of materials. In choosing a suitable material or materials, one characteristic of interest is mechanical strength. In various aspects, the housing 140 may be fabricated from various materials, categories of materials, and/or combinations of materials. In some aspects, signal amplification elements as described in U.S. patent application Ser. No. 12/238,345 (the disclosure of which is herein incorporated by reference) are modified to be used for this element.

In one embodiment, the capacitive plate 160 is configured to make direct contact with tissue and body fluid when the microstimulator 100 is implanted in a body. As such, the capacitive plate 160 of the microstimulator 100 may be configured as a stimulation element, for example where the capacitive plate 160 is configured as a single exposed electrode.

Referring now to the microstimulator 100 of FIG. 1, the integrated circuit 110 includes a number of distinct functional blocks, i.e., modules. In some aspects, the circuits include at least the following functional blocks: a power extraction functional block; an energy storage functional block; a sensor functional block; a communication functional block; a device configuration functional block; a rectifier functional block; etc. The integrated circuits 110 may be configured to perform one or more of the following functions: rectify a received radiofrequency signal to clock, data and power; connect the electrode of the device to either positive or negative voltage and connect the inner capacitive plate to either positive or negative voltage; communicate with an external power source and/or programming device; store information; etc. The integrated circuit 110 is also configured to activate the microstimulator 100 to apply an electrical stimulation to a target tissue, such as by reversing the polarities of the capacitor plate 130 and the capacitor plate 160 to apply an electrical stimulation pulse to a target tissue.

In various aspects, the integrated circuit 110 may include any or all of the functional blocks, all of which may be present in a single integrated circuit. By single integrated circuit is meant a single circuit structure that includes all of the different desired functional blocks. In these types of structures, the integrated circuit may be a monolithic integrated circuit that is a miniaturized electronic circuit which may be made up of semiconductor and passive components that have been manufactured in the surface of a thin substrate of a semiconductor material. Sensors of the invention may also include integrated circuits that are hybrid integrated circuits, which are miniaturized electronic circuits constructed of individual semiconductor devices, as well as passive components, bonded to a substrate or circuit board.

The microstimulators according to the teaching of the present invention may include a one or more components, such as the integrated circuit and the inductive power source, hermetically sealed in a hermetic sealing structure, such as the housing 140. Hermetic sealing structures are structures that seal the component or components of interest from the implanted environment so that the microstimulator maintains functionality, at least for the intended lifespan of the device. The nature of the hermetic sealing structure may vary, so long as it maintains the functionality of the microstimulator in the implanted environment for the desired period of time, such as one day or longer, one week or longer, one month or longer, one year or longer, five years or longer, ten years or longer, twenty-five years or longer, forty years or longer. The hermetic sealing structure is configured to protect certain components of the microstimulator, such as the integrated circuit and the inductive power source, such that the protected components remain functional for extended periods of time when implanted in a living body. The hermetic sealing structure may include at least one barrier structure, such as a thick layer of material, where the structure may be configured to provide a cavity that can house one or more components of interest. Alternatively, the structure may be configured to conform to the configuration of the one or more components that are to be sealed by the structure, such that no cavity is defined by the structure and the component or components sealed thereby. The barrier is one that is sufficient to prevent passage of a critical amount of one or more molecules of interest. Molecules of interest include water molecules, as well as body-associated ions. The critical amount whose passage is prevented is an amount that adversely affects the functioning (such as by corrosion) of the sealed components over the intended lifetime of the sensor.

In some aspects, the hermetic sealing structure is a conformal, void-free sealing layer, where the sealing layer is present on at least a portion of the outer surface of the integrated circuit component (described above). In some aspects, this conformal, void-free sealing layer may be present on substantially all of the outer surfaces of the integrated circuit component. Alternatively, this conformal, void-free sealing layer may be present over only some of the surfaces of the integrated circuit, such as over only one surface or even just a portion of one surface of the integrated circuit component. As such, some sensors have an integrated circuit component completely encased in a conformal, void free sealing layer. Other sensors are configured such that only the top surface of an integrated circuit component is covered with the conformal, void-free sealing layer.

The conformal, void-free sealing layer may be a “thin-film” coating, in that its thickness is such that it does not substantially increase the total volume of the integrated circuit structure with which it is associated, where any increase in volume of the structure that can be attributed to the layer may be 10% or less, such as 5% or less, including 1% or less by volume. In some aspects, the sealing layer has a thickness in a range from 0.1 to 10.0 μm, such as in a range from 0.3 to 3.0 μm thick, and including in a range 1.0 μm thick.

The sealing layer may be produced on the component to be sealed thereby using any of a number of different protocols, including but not limited to planar processing protocols, such as plasma-enhanced-chemical-vapor deposition, physical-vapor deposition, sputtering, evaporation, cathodic-arc deposition, low-pressure chemical-vapor deposition, etc.

Additional description of conformal, void-free sealing layers that may be employed for sensors of the invention is provided in PCT application serial no. PCT/US2007/009270 published under publication no. WO/2007/120884, the disclosure of which is herein incorporated by reference.

Also of interest as hermetic sealing structures are corrosion-resistant holders having at least one conductive feed-through and a sealing layer, where the sealing layer and holder are configured to define a hermetically sealed container that encloses the integrated circuit component, such as the housing 140 of FIG. 1. The conductive feed-through may be a metal, such as platinum, iridium etc., an alloy of metal and a semiconductor, a nitride, a semiconductor or some other convenient material. In some aspects, the corrosion-resistant holder comprises silicon or a ceramic. While dimensions may vary, the corrosion-resistant holder may have walls that are at least 1 μm thick, such as at least 50 μm thick, where the wall thickness may range from 1 to 125 μm, including from 25 to 100 μm. The sealing layer may be metallic, where metals of interest include noble metals and alloys thereof, such as platinum and platinum alloys. Dimensions of the sealing layer may also vary, ranging in some aspects from 0.5 μm thick or thicker, such as 2.0 μm thick or thicker, and including 20 μm thick or thickness, where sealing layer thicknesses may range from 0.5 to 100 μm, such as from 1 to 50 μm. In certain configurations, the structure further includes an insulative material present in the hermetically sealed volume. In some cases, the hermetically sealed volume ranges from 1 pl. to 1 ml.

In some aspects, the in-vivo corrosion-resistant holder is a structure, such as the housing 140, configured to hold an integrated circuit component such that the integrated circuit component is bounded on all but one side by the walls of the holder. For example, the holder 140 may include side walls and a bottom, where the holder may have a variety of different configurations as long as it contains the integrated circuit component in a manner such that the component is held in a volume bounded on all but one side. Accordingly, the shape of the holder may be square, circular, ovoid, rectangular, or some other shape as desired.

Additional description of corrosion resistant holders that may be employed for sensors of the invention is provided in PCT application serial no. PCT/US2005/046815 published under publication no. WO/2006/069323, the disclosure of which is herein incorporated by reference.

The housing 140 may include a number of different features depending on the particular design of a given microstimulator. For example, the housing 140 may include attachment elements for securing the microstimulator 100 to tissue, such as suture hole, barb, or other structure that may find use, either alone or in conjunction with another element.

The housing 140 may also serve as a depot for one or more desired bioactive agents which may be included for a variety of purposes. Bioactive agents that may be incorporated into the electrically insulating element (where the element may be configured to release the incorporated bioactive agents) include but are not limited to: anti-inflammatory agents, tissue growth inhibitory agents, anti-microbial agents, chemotherapeutic agents, etc.

Referring now to FIG. 2, another configuration for a microstimulator in accordance with the teachings of the present invention is shown. In FIG. 2, microstimulator 200 includes a pellet configuration. Hermetically sealed inside the microstimulator 200 are an integrated circuit 210 and a coil 220, where the components are as described above. Also shown are a single exposed electrode 260 and a capacitor plate 230. The microstimulator 200 has a small form factor, such that they occupy a small volume of space. In some aspects, the microstimulators occupy a volume of 8 mm³ or less, such as 4 mm³ or less, including 1 mm³ or less. In some aspects, the microstimulators have a capsule configuration, in which active components are sealed in a container having a defined spaced. For example, microstimulators of the invention may include a container structure as described in U.S. Pat. No. 6,871,099.

A microstimulator may operate independently, or in a coordinated manner with other implanted devices, or with external devices. In addition, a microstimulator may incorporate a pain sensor, which it may then use to control stimulation parameters in a closed-loop manner. According to one embodiment of the invention, the sensing and stimulating means may be incorporated into a single microstimulator. According to one embodiment of the invention, a sensing means communicates sensed information to at least one microstimulator with stimulating means. According to one embodiment of the invention, a microstimulator operates independently. According to another embodiment of the invention, a microstimulator operates in a coordinated manner with other microstimulator(s), other implanted device(s), or other device(s) external to the patient's body. For instance, a microstimulator may control or operate under the control of another implanted microstimulator(s), of other implanted device(s), or other device(s) external to the patient's body. A microstimulator may communicate with other implanted microstimulators, other implanted devices, and/or devices external to a patient's body via, e.g., an RF link, an ultrasonic link, a thermal link, or an optical link. Specifically, a microstimulator may communicate with an external remote control (e.g., patient and/or physician programmer) that is capable of sending commands and/or data to a microstimulator and that is preferably capable of receiving commands and/or data from a microstimulator.

A view of a microstimulator that is configured to operate in conjunction with another device, such as another microstimulator, is shown in FIG. 3. In FIG. 3, microstimulator 300 includes an integrated circuit 310 and one a capacitive plate 320. The capacitive plate 320 is configured as an electrode that is part of a hermetic sealing structure 330. The microstimulator 300 is configured to operate in conjunction with other similar microstimulators, such that during use first capacitive plate 320 is employed in conjunction with an analogous capacitive plate on a separated microstimulator.

Also provided are systems that include one more neural microstimulators as described above. The systems of the invention may be made up solely of microstimulators, or they may include one or more additional types of components, such as internal or external receivers, control units, etc. Some systems of the invention include one or more microstimulators and a personal health receiver, e.g., as described in PCT/US2008/052845 published as WO/2008/095183 and PCT/US2006/016370 published as WO/2006/116718; the disclosures of which are herein incorporated by reference. In some aspects a receiver as described in these publications that includes an electrode is employed in conjunction with a microstimulator of the invention to deliver a stimulation pulse to a target tissue. In some aspects, systems of invention include one or more microstimulators configured to respond to a signal emitted by an ingestible event marker, e.g., as described in PCT/US2008/052845 published as WO/2008/095183, the disclosure of which is herein incorporated by reference.

Also provided are methods of using the microstimulators of the invention. The methods of the invention may include: providing one or more microstimulators of the invention, e.g., as described above, that includes implantable electrical stimulation lead of the invention, as described above. The one or more microstimulators may be implanted in a suitable subject using any convenient approach. Following implantation, the one or more microstimulators may be employed to as desired to treat a condition of interest.

During use, a health care professional, such as a physician or other clinician, may select values for a number of programmable parameters in order to define the stimulation therapy to be delivered to a patient. For example, the health care professional may select a voltage or current amplitude and pulse width for a stimulation waveform to be delivered to the patient, as well as a rate at which the pulses are to be delivered to the patient and a duty cycle. The health care professional may also select as parameters particular microstimulators within a set of distributed microstimulators to be used to deliver the pulses, and the polarities of the selected electrodes. A group of parameter values may be referred to as a program in the sense that they drive the neurostimulation therapy to be delivered to the patient.

A health care professional may select parameter values for a number of programs to be tested on a patient during a programming session. The programming device directs the implantable neurostimulator implanted in the patent to deliver neurostimulation according to each program, and the health care professional collects feedback from the patient, e.g., rating information, for each program tested on the patient. The health care professional then selects one or more programs for long-term use by the implantable neurostimulator based on the rating information.

Implantable microstimulators of the invention find use in any application where electrical stimulation of target tissue in a patient is desired. Implantable microstimulators of the invention may be employed in a variety of different applications. Examples of applications include the use of the devices and systems to deliver neurostimulation therapy to patients to treat a variety of symptoms or conditions such as chronic pain, tremor, Parkinson's disease, epilepsy, incontinence, or gastroparesis. Implantable neurostimulators may deliver neurostimulation therapy in the form of electrical pulses via leads that include electrodes. To treat the above-identified symptoms or conditions, for example, the electrodes may be located proximate to the spinal cord, pelvic nerves, or stomach, or within the brain of a patient. Microstimulators of the invention find use in applications and methods as described in U.S. Pat. Nos. 5,193,539; 5,193,540; 5,312,439; 5,324,316; 5,405,367; 6,051,016, 6,871,099 and published application no. 20040015205; the disclosures of which are herein incorporated by reference.

Also provided are kits that include the one or more microstimulators. Kits may also include receivers, controllers or other components as desired, including those described above. In various aspects of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular aspects described, as such may vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual aspects described and illustrated herein has discrete components and features which may be readily separated from or combined with the features of any of the other several aspects without departing from the scope or spirit of the present invention. Any recited method can be carried out in the order of events recited or in any other order which is logically possible.

1. A system for stimulating a target site, the system comprising:
a hermetically sealed housing wherein the shape of the housing provides two distal locations to allow for focused stimulation of the target site;
an integrated circuit secured within the housing for controlling stimulation of the target site;
a power source secured within the housing and in electrical communication with the integrated circuit for powering the circuit;
a capacitive plate secured at the perimeter of the housing and electrically coupled to the circuit, wherein the capacitive plate includes a dielectric layer that separates the capacitive plate from body fluid when the system is implanted in a body; and
a conductive element secured to the outer surface of the housing distal from the capacitive plate, wherein the conductive element is electrically coupled to the circuit and the conductive element is in direct contact with the target site;
wherein the housing is made of non-conductive material and the circuit controls the polarity of the conductive element relative to the capacitive plate to stimulate the target site.

2-3. (canceled)

4. The system of claim 1, wherein the housing defines a hole therein for securing the housing to the target site.

5. An implantable microstimulator, wherein the implantable microstimulator comprises:
a hermetically sealed structure comprising therein:
an integrated circuit; and
an inductive power source configured to receive power from a source external to the microstimulator;
wherein the hermetic sealing structure comprises a conformal, void-free sealing layer;
a first capacitive plate electrically coupled to the integrated circuit and configured as a single exposed electrode; and
a second capacitive plate conductively coupled to the integrated circuit, wherein the microstimulator is configured such that when the microstimulator is implanted in a body, the first and second capacitive plate together with body fluid make up an electrolytic capacitor.

6-8. (canceled)

9. The implantable microstimulator of claim 5, wherein the second capacitive plate comprises a surface including:
a high surface area; and
a thin dielectric layer.

10. The implantable microstimulator of claim 5, wherein the microstimulator occupies a volume of 8 mm³ or less.

11. The implantable microstimulator of claim 5, wherein the microstimulator occupies a volume of 4 mm³ or less.

12. The implantable microstimulator of claim 5, wherein the microstimulator occupies a volume of 1 mm³ or less.

13-16. (canceled)
